SEQUENCE LISTING (15 sequences)

SEQ ID NO: 1 - protein, 16 aa, synthetic construct
RSTKSLLHSNGDTYLY

SEQ ID NO: 2 - protein, 7 aa, synthetic construct
RMSNLAP

SEQ ID NO: 3 - protein, 6 aa, synthetic construct
TGYNMN

SEQ ID NO: 4 - protein, 17 aa, synthetic construct
NINPYFGSPNYNQKFKG

SEQ ID NO: 5 - protein, 11 aa, synthetic construct
RQLGLGDTMDY

SEQ ID NO: 6 - protein, 11 aa, synthetic construct
KASQDVSTAVG

SEQ ID NO: 7 - protein, 7 aa, synthetic construct
WASTRHT

SEQ ID NO: 8 - protein, 4 aa, synthetic construct
QQHY

SEQ ID NO: 9 - protein, 6 aa, synthetic construct
TDFYIS

SEQ ID NO: 10 - protein, 17 aa, synthetic construct
WIYPLDDHTKYNEKFKG

SEQ ID NO: 11 - protein, 8 aa, synthetic construct
IYGYAMDY

SEQ ID NO: 12 - protein, 113 aa, synthetic construct
LEYPFTFGAGTKLELKDIVMTQTASSVYVTPGESVSISCRSTKSLLHSNGDTYLYWFLQRPGQSPQLLIYRMSNLAPGVPDRFSGGGSGTAFTLRISRVEAEDVGVYYCMQHI

SEQ ID NO: 13 - protein, 125 aa, synthetic construct
CEIQLQQSGALELVRPGTSVAKISCMASGYISFTGYNMNWRVRQSHGRSLEWIGNINPYFGSPNYNQKFKGKATLTLDRSSNTAYMQLNRVTSEDSAVYYCASRQLGLGDTMDYWGQGTSVTVSS

SEQ ID NO: 14 - protein, 109 aa, synthetic construct
LEYPFTFGGGTKLEIKDIVMTQSHKFMSTSVGDRVTITCKASQDVSTAVGWYQQKPGQSPKLLIYWASTRHTGVPDRFTGSRFGTDYTLTISSVQAEDLALYYCQQHYI

SEQ ID NO: 15 - protein, 122 aa, synthetic construct
CQIQLQQSGPLELGKPGASVAKISCKASDYITFTDFYISWRVKQRPGQGLEWIGWIYPLDDHTKYNEKFKGKATLTVDISSNTVYMQLSSLTSEDSAVYFCARIYGYAMDYWGQGTSVTVSS

(19) United States
(12) Patent Application Publication
(10) Pub. No.: US 2024/0117070 A1
(43) Pub. Date: Apr. 11, 2024

(54) RECOMBINANT ANTIBODIES, KITS COMPRISING THE SAME, AND USES THEREOF

(71) Applicant: CLAIR CO., LTD., Taipei City (TW)

(72) Inventors: Jen-Chieh YU, Taipei City (TW); Wen-Chi HUANG, Taipei City (TW)

(21) Appl. No.: 18/376,628

(22) Filed: Oct. 4, 2023

(30) Foreign Application Priority Data
Oct. 10, 2022 (CN) 202211235487.2

(51) Int. Cl. C07K 16/30; G01N 33/574

(52) U.S. Cl. CPC C07K 16/3084; G01N 33/57423; C07K 2317/565; G01N 2800/56

(57) ABSTRACT

Disclosed herein are recombinant antibodies or the fragment thereof for detecting ganglioside GM2 activator (GM2A) protein in vitro. Also disclosed herein are kits and methods for determining whether a subject has or is at risk of developing lung cancer with the aid of the present recombinant antibodies.

### CROSS-REFERENCES TO RELATED APPLICATIONS

This application claims priority and the benefit of China Patent Application No. 202211235487.2, filed Oct. 10, 2022, the entireties of which is incorporated herein by reference.

### SEQUENCE LISTING XML

The present application is being filed along with a Sequence Listing XML in electronic format. The Sequence Listing XML is provided as an XML file entitled P4284-US_segeunce listing, created Sep. 7, 2023, which is 15 Kb in size. The information in the electronic format of the Sequence Listing XML is incorporated herein by reference in its entirety.

### BACKGROUND OF THE INVENTION

### 1. Field of the Invention

The present disclosure relates to the field of disease diagnosis. More particularly, the disclosed invention relates to a recombinant antibody and its use for determining the expression level of ganglioside GM2 activator protein (GM2A) in a sample of a subject for diagnosing whether the subject is afflicted with lung cancer.

### 2. Description of Related Art

According to statistical data published by World Health Organization (WHO), lung cancer is the leading cause of cancer-related deaths on a global scale, as well as in China. Up to the year of 2022, lung cancer has accounted for 17.9% of newly reported cancer cases in China. This emphasizes the increasing health threat presented by lung cancer to the general population.

Lung cancer may be classified into two types: Small Cell Lung Cancer (SCLC) and Non-Small Cell Lung Cancer (NSCLC), constituting 15% and 85% of lung cancer cases, respectively. Regardless of the lung cancer types, existing clinical diagnostic approaches mainly involve invasive techniques such as sputum cytology, imaging scans, and bronchoscopy. However, these invasive techniques are limited by their accuracy and sensitivity in assessing the progression of cancer.

Due to the lack of effective tools for early diagnosis and the inability to treat advanced-stage cancer, the 5-year survival rate for lung cancer patient is a scantly 15%. Consequently, for lung cancer patients, early diagnosis followed by appropriate treatment may significantly enhance both survival rates and prognostic outcomes.

In view of the foregoing, there exists in the related art a need for an effective way for the diagnosis of lung cancer, particularly, the lung cancer at an early stage.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the present disclosure is directed to a recombinant GM2A antibody, which comprises a light chain variable (VL) region and a heavy chain variable (VH) region, wherein the VL region comprises a first complementarity determining region (CDR-L1), a second complementarity determining region (CDR-L2), and a third complementarity determining region (CDR-L3), and the VH region comprises a first complementarity determining region (CDR-H1), a second complementarity determining region (CDR-H2), and a third complementarity determining region (CDR-H3).

According to some embodiments of the present disclosure, the CDR-L1 of the VL region comprises the amino acid sequence of SEQ ID NO: 1; the CDR-L2 of the VL region comprises the amino acid sequence of SEQ ID NO: 2; the CDR-L3 of the VL region comprises the amino acid sequence of MQH; the CDR-H1 of the VH region comprises the amino acid sequence of SEQ ID NO: 3; the CDR-H2 of the VH region comprises the amino acid sequence of SEQ ID NO: 4, and the CDR-H3 of the VH region comprises the amino acid sequence of SEQ ID NO: 5. According to some preferred embodiments of the present disclosure, the VL region comprises an amino acid sequence at least 85% identical to SEQ ID NO: 12, and the VH region comprises an amino acid sequence at least 85% identical to SEQ ID NO: 13. In some working examples, the VL region and the VH region respectively have the amino acid sequences of SEQ ID NOs: 12 and 13.

According to some embodiments of the present disclosure, the CDR-L1 of the VL region comprises the amino acid sequence of SEQ ID NO: 6; the CDR-L2 of the VL region comprises the amino acid sequence of SEQ ID NO: 7; the CDR-L3 of the VL region comprises the amino acid sequence of SEQ ID NO: 8; the CDR-H1 of the VH region comprises the amino acid sequence of SEQ ID NO: 9; the CDR-H2 of the VH region comprises the amino acid sequence of SEQ ID NO: 10, and the CDR-H3 of the VH region comprises the amino acid sequence of SEQ ID NO: 11. According to some preferred embodiments of the present disclosure, the VL region comprises an amino acid sequence at least 85% identical to SEQ ID NO: 14, and the VH region comprises an amino acid sequence at least 85% identical to SEQ ID NO: 15. In some working examples, the VL region and the VH region respectively have an amino acid sequences of SEQ ID NOs: 14 and 15.

Also disclosed herein is a kit for detecting the expression level of GM2A in a biological sample. The kit of the present disclosure comprises a first recombinant antibody, a second recombinant antibody, and a container containing the first and second recombinant antibodies. In the present disclosure, the first recombinant antibody and the second recombinant antibody are respectively selected from the recombinant antibodies set forth in the first aspect. In accordance with some embodiments of the present disclosure, the VL region of the first recombinant antibody comprises the CDR-L1, the CDR-L2, and the CDR-L3; the VH region of the second recombinant antibody comprises the CDR-H1, the CDR-H2, and the CDR-H3, wherein the CDR-L1, the CDR-L2, the CDR-H1, the CDR-H2, and the CDR-H3 respectively comprise the amino acid sequences of SEQ ID NOs: 1, 2, 3, 4, and 5, and the CDR-L3 comprises the amino acid sequence of MQH; and the CDR-L1, the CDR-L2, the CDR-L3 of the VL region of the second recombinant antibody respectively comprise the amino acid sequences of SEQ ID NOs: 6, 7, and 8, and the CDR-H1, the CDR-H2, and the CDR-H3 of the VH region of the second recombinant antibody respectively comprise the amino acid sequences of SEQ ID NOs: 9, 10, and 11.

According to some embodiments of the present disclosure, the VL region and the VH region of the first recombinant antibody comprise amino acid sequences at least 85% identical to SEQ ID NOs: 12 and 13, respectively. The VL region and the VH region of the second recombinant antibody comprise amino acid sequences at least 85% identical to SEQ ID NOs: 14 and 15, respectively.

According to one embodiment of the present disclosure, the VL region of the first recombinant antibody has an amino acid sequence of SEQ ID NO: 12, and the VH region of the first recombinant antibody has an amino acid sequence of SEQ ID NO: 13; and the VL region of the second recombinant antibody has an amino acid sequence of SEQ ID NO: 14, and the VH region of the second recombinant antibody has an amino acid sequence of SEQ ID NO: 15.

Another aspect of the present disclosure is directed to a method for determining whether a subject has a lung cancer by use of a biological sample isolated from the subject (i.e., an isolated biological sample). The method comprises the following steps: (a) detecting the level of GM2A protein in the isolated biological sample by use of the recombinant antibodies or the kit of the present disclosure; and (b) comparing the level of GM2A protein in the isolated biological sample of step (a) with that in a control sample, wherein the subject has the lung cancer when the detected level of GM2A protein in the biological sample is higher than that in the control sample.

According to some embodiments of the present disclosure, the biological sample isolated from the subject is a urine sample.

According to some embodiments of the present disclosure, the subject is a human.

According to some embodiments of the present disclosure, the lung cancer is at an early stage.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a plotted graph illustrating the calibration results of detection of the GM2A protein standard using the present recombinant antibodies according to one embodiment of the present disclosure.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example.

However, the same or equivalent functions and sequences may be accomplished by different examples.

### 1. Definition

For convenience, certain terms employed in the specification, examples and appended claims are collected here. Unless defined otherwise, all technical and scientific terms used herein have the same meaning as commonly understood by one of the ordinary skill in the art to which this invention belongs. Also, unless otherwise required by context, it will be understood that singular terms shall include plural forms of the same and plural terms shall include the singular. Specifically, as used herein and in the claims, the singular forms “a”, “and”, and “the” are used herein to include plural referents unless the context clearly dictates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “antibody” is used in the broadest sense and specifically covers monoclonal antibodies (including full length monoclonal antibodies), polyclonal antibodies, multi-specific or multivalent antibodies (e.g., bi-specific antibodies), and antibody fragments so long as they exhibit the desired biological activity. The term “antibody fragment” or “a fragment of an antibody” refers to a portion of a full-length antibody, generally the antigen binding or variable region (i.e., VL and VH regions) of a full-length antibody. Examples of the antibody fragment include fragment antigen-binding (Fab), Fab′, F(ab′)2, single-chain variable fragment (scFv), diabody, linear antibody, single-chain antibody molecule, and multi-specific antibody formed from antibody fragments.

The term “complementarity determining region (CDR)” as used herein refers to the hypervariable region of an antibody molecule that forms a surface complementary to the 3-dimensional surface of a bound antigen. In general, an antibody consists of heavy and light chains, each containing three CDRs (designated as CDR-1, CDR-2, and CDR-3 from the N-terminus to the C-terminus). Therefore, includes a total of six CDRs that comprise three CDRs from the variable region of a heavy chain (i.e., CDR-H1, CDR-H2, and CDR-H3), and three CDRs from the variable region of a light chain (i.e., CDR-L1, CDR-L2, and CDR-L3). The amino acid residues of CDRs are in close contact with bound antigen.

The term “variable region” of an antibody as used in herein refers to the amino-terminal regions of the heavy or light chains of the antibody, as opposed to the “constant region.” These regions are generally the most variable parts of an antibody and contain the antigen-binding sites. The term “variable” refers to the fact that certain portions of the variable regions differ extensively in sequence among antibodies, and are used in the binding and specificity of each particular antibody for its particular antigen. However, the variability is not evenly distributed throughout the variable regions of antibodies. It is concentrated in three segments called complementarity-determining regions (CDRs) or hypervariable regions both in the light-chain and the heavy-chain variable regions. The more highly conserved portions of variable regions are called the framework (FR). The variable regions of native heavy and light chains each comprises four FR regions, largely adopting a beta-sheet configuration, connected by three CDRs, which form loops connecting, and in some cases forming part of, the beta-sheet structure. The CDRs in each chain are held together close proximity by the FR regions, and with the CDRs from the other chain, contribute to the formation of the antigen-binding site of antibodies. The constant regions are not involved directly in binding an antibody to an antigen, but exhibit various effector functions, such as participation of the antibody in antibody-dependent cellular toxicity.

“Percentage (%) sequence identity” with respect to any amino acid sequence identified herein is defined as the percentage of amino acid residues in a candidate sequence that are identical with the amino acid residues in the specific reference sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two amino acid sequences was carried out by computer program Blastp (protein-protein BLAST) provided online by Nation Center for Biotechnology Information (NCBI). The percentage sequence identity of a given sequence A to a subject sequence B (which can alternatively be phrased as a given sequence A that has a certain % sequence identity to a given sequence B) is calculated by the formula as follows:

X/Y×100%

where X is the number of amino acid residues scored as identical matches by the sequence alignment program BLAST in that program's alignment of A and B, and where Y is the total number of amino acid residues in the subject sequence B.

As discussed herein, minor variations in the amino acid sequences of antibodies are contemplated as being encompassed by the presently disclosed and claimed inventive concept(s), providing that the variations in the amino acid sequence maintain at least 85% sequence identity, such as at least 85%, 86%, 87%, 88%, 89%, 90%, 91%, 92%, 93%, 94%, 95%, 96%, 97%, 98% and 99% sequence identity. Antibodies of the present disclosure may be modified specifically to alter a feature of the peptide unrelated to its physiological activity. For example, certain amino acids can be changed and/or deleted without affecting the physiological activity of the antibody in this study (i.e., the ability of binding to ganglioside GM2 activator (GM2A) protein).

Conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the peptide derivative. Fragments or analogs of antibodies can be readily prepared by those of ordinary skill in the art. Preferred amino- and carboxyl-termini of fragments or analogs occur near boundaries of functional regions.

The term “subject” refers to a mammal including the human species that can be subjected to the recombinant antibodies, kits and/or methods of the present disclosure. The term “subject” is intended to refer to both the male and female gender unless one gender is specifically indicated.

The term “isolated biological sample” or “isolated sample” as interchangeably used herein refers to any biological sample isolated from an organism (including living or once-living organic entities). As long as manipulation or additional treatment is performed on the biological sample, it is considered distinct and dependent from the individual. An “isolated sample” can encompass liquid samples (such as serum samples and urine samples) and tissue samples (such as tissue sections and tissue biopsies) isolated from the organism; cell samples (for example, cytological smears like Pap smears or blood smears) or cells obtained through microdissection; or cellular components, fragments, or organelles (obtained, for instance, by lysing cells and separating their constituents through centrifugation or other means). Specific examples of isolated samples include whole blood, serum, plasma, urine, saliva, cerebrospinal fluid, synovial fluid, pleural fluid, peritoneal fluid, lymphatic fluid, or combinations thereof.

The term “diagnosis” or “diagnose” as interchangeably used herein refers to a method by which a person skilled in the art can estimate and/or determine the probability (likelihood) of a subject having a given disease or condition. This type of diagnosis or determination does not imply 100% accuracy, but rather aims to compare the general state presented by an individual against a certain ailment-free (or non-disease) condition, providing a reference result with higher reliability. Numerous biomarkers can indicate the presence or absence of various conditions. Clinical practitioners can rely solely on the results of biomarker analysis or use them in combination with other clinical indications to achieve a diagnosis. Consequently, measurement values significantly higher or lower than those of healthy individuals indicate a greater likelihood of the individual having the disease, relative to the norm.

According to TNM staging system, the course of a lung cancer is classified into five stages, which are Stage 0, Stage I, Stage II, Stage III, and Stage IV, based on the number and the size of tumors, tumor metastasis, and the overall performance status of a patient. In Stage 0, the tumor locates in the top lining of the lung or bronchus and has not spread; in Stage I, the tumor size is developed to 1 to 3 cm. The term “an early stage” of a lung cancer as used herein refers to Stage 0 to Stage I of the lung cancer.

### 2. Description of the Invention

The present disclosure is at least based on the discovery of the level of the GM2A protein in a biological sample may serve as a biomarker for the detection of lung cancer. Thus, the present disclosure aims to provide a recombinant antibody that specifically binds to the GM2A protein, and a kit comprising the recombinant antibody. Also disclosed herein is a use of the present recombinant antibody for detecting GM2A protein level in an isolated biological sample, which in turn helps in determining whether the subject is affiliated with lung cancer or not.

2.1 The Recombinant Antibody

The first aspect of the present disclosure is directed to two recombinant antibodies respectively designated as anti-GM2A-I and anti-GM2A-II, in which both antibodies are IgG antibodies. According to embodiments of the present disclosure, the anti-GM2A-I or the anti-GM2A-II independently comprises a light chain variable (VL) region and a heavy chain variable (VH) region, wherein the VL region comprises three complementarity determining regions (i.e., CDR-L1, CDR-L2, and CDR-L3), and the VH region comprises three CDRs, which are respectively CDR-H1, CDR-H2, and CDR-H3.

According to some embodiments of the present disclosure, the CDR-L1, and CDR-L2 of the recombinant anti-GM2A-I antibody respectively comprise the amino acid sequences of SEQ ID NOs: 1 and 2, while the CDR-L3 comprises the amino acid sequence of “MQH”; and the CDR-H1, CDR-H2, and CDR-H3 of the recombinant anti-GM2A-I antibody respectively comprise the amino acid sequences of SEQ ID NOs: 3, 4, and 5. Preferably, the VL region of the recombinant anti-GM2A-I antibody comprises the amino acid sequence at least 80% (i.e., 80%, 81%, 82%, 83%, 84%, 85%, 86%, 87%, 88%, 89%, 90%, 91%, 92%, 93%, 94%, 95%, 96%, 97%, 98%, 99% or 100%) identical to SEQ ID NO: 12, and the VH region of the recombinant anti-GM2A-I antibody comprises the amino acid sequence at least 80% (i.e., 80%, 81%, 82%, 83%, 84%, 85%, 86%, 87%, 88%, 89%, 90%, 91%, 92%, 93%, 94%, 95%, 96%, 97%, 98%, 99% or 100%) identical to SEQ ID NO: 13.

According to some embodiments of the present disclosure, the CDR-L1, CDR-L2, and CDR-L3 of the recombinant anti-GM2A-II antibody respectively comprise the amino acid sequences of SEQ ID NOs: 6, 7, and 8; and the CDR-H1, CDR-H2, and CDR-H3 of the recombinant anti-GM2A-II antibody respectively comprise the amino acid sequences of SEQ ID NOs: 9, 10, and 11. Preferably, the VL region of the recombinant anti-GM2A-II antibody comprises the amino acid sequence at least 80% identical to SEQ ID NO: 14, and the VH region of the recombinant anti-GM2A-II antibody comprises the amino acid sequence at least 80% identical to SEQ ID NO: 15. According to the preferred embodiment, the VL and VH regions of the recombinant anti-GM2A-II antibody respectively comprise the amino acid sequences at least 85% identical to SEQ ID NOs: 14 and 15. More preferably, the VL and VH regions of the recombinant anti-GM2A-II antibody respectively comprise the amino acid sequences at least 90% identical to SEQ ID NOs: 14 and 15. Even more preferably, the VL and VH regions of the recombinant anti-GM2A-II antibody respectively comprise the amino acid sequences at least 95% identical to SEQ ID NOs: 14 and 15. In one working example of the present disclosure, the VL region of the recombinant anti-GM2A-II antibody has the amino acid sequence of SEQ ID NO: 14, and the VH region of the recombinant anti-GM2A-II antibody has the amino acid sequence of SEQ ID NO: 15.

The recombinant antibodies of the present disclosure can be produced by tools and approaches well-known in the art. Examples of production method commonly used in the art include, but are not limited to, hybridoma, phage display, proteomic analysis, B cell sorting and cloning techniques, and a combination thereof. In practice, specific antibodies required can be prepared through the combination of proteomic analysis and single B cell sorting and cloning. Typically, after the target protein (such as the GM2A protein in the present disclosure) is selected, the amino acid sequence, the secondary and tertiary structures of the protein can be analyzed by using conventional mass spectrometry and sequencing approaches. Meanwhile, animals (such as rabbits or rodents) can be immunized to induce antibody production in their bodies. Based on the deciphered amino acid sequence and protein structure, proteomic engineering can be employed to reverse-engineer the antibodies obtained from the animal, thereby screening for antibodies that exhibit the desired target characteristics. According to some embodiments of the present disclosure, mass spectrometry is used to analysis the amino acid sequence and structure of GM2A protein (such as sequence number: 5). Rabbits are immunized concurrently, and thousands of B-cells produced in the rabbits are sorted and separated using flow cytometry. Those B-cell lines are individually subjected to cultivation and expansion for a period of time, then subjected to sequencing (e.g., single-cell sequencing) to screen genes encoding the light and heavy chains of the antibodies. After amplified by using nucleic acid amplification, the antibody genes are cloned into expression vectors, allowing producing recombinant antibodies in mammalian cell systems. Monoclonal antibodies having higher binding affinity for GM2A protein are obtained after purification and analysis, thereby producing the present recombinant antibodies, anti-GM2A-I and anti-GM2A-II.

The sequence identifiers corresponding to the CDR sequences (including CDR-L1, CDR-L2, CDR-L3, CDR-H1, CDR-H2 and CDR-H3) of the present recombinant antibodies (i.e., anti-GM2A-I and anti-GM2A-II) are summarized in Table 1.

TABLE 1
Sequence identifiers (SEQ ID NOs) corresponding
to the CDR sequences of specified antibodies

| | Sequence identifier (SEQ ID NO) | | | | | |
|---|---|---|---|---|---|---|
| | CDR- | CDR- | CDR- | CDR- | CDR- | CDR- |
| Antibody | L1 | L2 | L3 | H1 | H2 | H3 |
| anti-GM2A-I | 1 | 2 | MQH | 3 | 4 | 5 |
| anti-GM2A-II | 6 | 7 | 8 | 9 | 10 | 11 |

*Sequences with fewer than 4 amino acids are not numbered by sequence identifier and are directly depicted in Table 1.

According to some embodiments of the present disclosure, the VL domain and the VH domain of anti-GM2A-I and anti-GM2A-II antibodies respectively comprise the amino acid sequences as summarized in Table 2.

TABLE 2
Sequence identifiers corresponding to the
VL and VH sequences of specified antibodies

| | | Sequence identifier |
|---|---|---|
| Antibody | Domain | (SEQ ID NO) |
| anti-GM2A-I | VL | 12 |
| | VH | 13 |
| anti-GM2A-II | VL | 14 |
| | VH | 15 |

As would be appreciated, the sequence (e.g., the framework sequence) of the VL or VH domains may vary (e.g., being substituted by conserved or non-conserved amino acid residues) without affecting the binding affinity and/or specificity of the present antibody. Preferably, the sequence(s) of the VL and VH domains is/are conservatively substituted by one or more suitable conservative amino acid residue(s) with similar properties; for example, the substitution of leucine (an nonpolar amino acid residue) by isoleucine, alanine, valine, proline, phenylalanine, or tryptophan (another nonpolar amino acid residue); the substitution of aspartate (an acidic amino acid residue) by glutamate (another acidic amino acid residue); or the substitution of lysine (an basic amino acid residue) by arginine or histidine (another basic amino acid residue). According to one preferred embodiment, the VL domain and VH domain of the recombinant anti-GM2A-I antibody may respectively comprise amino acid sequences at least 85% identical to SEQ ID NOs: 12 and 13. Preferably, the VL domain and VH domain of the recombinant anti-GM2A-I antibody may respectively comprise the amino acid sequences at least 90% identical to SEQ ID NOs: 12 and 13. More preferably, the VL domain and VH domain of the recombinant anti-GM2A-I antibody may respectively comprise the amino acid sequences at least 95% identical to SEQ ID NOs: 12 and 13. In one working embodiment, the VL domain of the recombinant anti-GM2A-I antibody has the amino acid sequence of SEQ ID NO: 12, and the VH domain of the recombinant anti-GM2A-I antibody has the amino acid sequence of SEQ ID NO: 13. According to another preferred embodiment, the VL domain and VH domain of the recombinant anti-GM2A-II antibody may comprise the amino acid sequences at least 85% identical to SEQ ID NOs: 14 and 15, respectively. Preferably, the VL domain and VH domain of the recombinant anti-GM2A-II antibody may comprise the amino acid sequences at least 90% identical to SEQ ID NOs: 14 and 15, respectively. More preferably, the VL domain and VH domain of the recombinant anti-GM2A-II antibody may comprise the amino acid sequences at least 95% identical to SEQ ID NOs: 14 and 15, respectively. In one working embodiment, the VL domain of the recombinant anti-GM2A-II antibody has the amino acid sequence of SEQ ID NO: 14, and the VH domain of the recombinant anti-GM2A-II antibody has the amino acid sequence of SEQ ID NO: 15.

Optionally, each recombinant antibody of the present disclosure may be conjugated with a reporter molecule or a nanoparticle. Exemplary reporter molecules include, but are not limited to, acridine orange, acridine yellow, alkaline phosphatase (AP), auramine, benzoxadiazole, bilirubin, biotin, blue fluorescent protein (BFP), 6′-carboxyfluorescein (FAM), cascade blue, cresyl violet, crystal violet, cyan fluorescent protein (CFP), cyanine, DNA probe, eosin, fluorescein, fluorescein isothiocyanate, glutathione-S-transferase (GST), green fluorescent protein (GFP), horseradish peroxidase (HRP), indocarbocyanine, malachite green, merocyanine, Nile blue, Nile red, nitrobenzoxadiazole, orotidine 5′-phosphate decarboxylase, oxacarbocyanine, peridinin chlorophyll, phycocyanin, phthalocyanine, porphine, protochlorophyll, pyridyloxazole, red fluorescent protein (RFP), rhodamine, thiacarbocyanine, thioredoxin (TRX), and yellow fluorescent protein (YFP). According to one working example, the recombinant antibody anti-GM2A-II is conjugated with HRP.

Accordingly, the recombinant antibodies of the present disclosure, anti-GM2A-I and anti-GM2A-II are capable of binding to GM2A specifically, therefore can serve as detection reagents for determining the level of GM2A protein in biological samples.

2.2 The Kit for Detecting GM2A Protein in Biological Samples

Another aspect of the present disclosure is directed to a kit for detecting GM2A protein in a biological sample. The kit comprises a first recombinant antibody, a second recombinant antibody, and a container configured to accommodate the first and second recombinant antibodies therein. According to some embodiments, the first and second recombinant antibodies are respectively anti-GM2A-I and anti-GM2A-II antibodies set forth in section 2.1. The kit of the present disclosure can be used to detect the level of GM2A protein in a biological sample using any of detection techniques well-known in the art (such as enzyme-linked immunosorbent assay (ELISA), flow cytometry, Western blotting, immunohistochemistry, and mass spectrometry). According to some embodiments of the present disclosure, one of the first and second recombinant antibodies serves as a capture antibody for capturing the GM2A protein in the sample, and the other one serves as a detection antibody for detecting the antibody-GM2A protein complex (i.e., the GM2A protein bound to the capture antibody) formed in ELISA.

According to working examples of the present disclosure, the first recombinant antibody (anti-GM2A-I) serves as the capture antibody; and the second recombinant antibody (anti-GM2A-II) serves as the detection antibody.

The container of the present kit in the present disclosure is equipped with a suitable packaging to accommodate the first and second recombinant antibodies therein. Examples of packaging suitable for use in the present kit include, but are not limited to, vials, bottles, jars, flexible packaging (e.g., sealed polyethylene terephthalate (PET) film or plastic bags), boxes, and the like. The kit may alternatively or optionally comprise additional components, such as buffers and interpretive information including usage instructions for using the present antibody or the fragment thereof to detect the GM2A protein in samples. The usage instructions provided by the present kit are typically in the form of written instructions on a label or package insert (such as a piece of paper included within the kit), or can be in the form of machine-readable operating instructions (such as a URL link to cloud-based data, a disk, or a CD). In some embodiments of the present disclosure, the invention provides articles of manufacture comprising contents of the kits as described above.

2.3 Diagnostic Method

The third aspect of the present invention is to provide a method for determining whether a subject has a lung cancer by using a biological sample isolated from the subject. The method comprises the steps of, detecting the level of GM2A protein in the biological sample by use of the present recombinant antibody or the present kit; and comparing the detected level of GM2A protein in the biological sample with that in a control sample (e.g., a biological sample of a healthy subject), wherein if the detected level of GM2A protein in the biological sample is higher than that of in the control sample, then the subject has the lung cancer.

According to embodiments of the present disclosure, the biological sample suitable for use in the present method is any biological sample that has been isolated from a living organism (including living or was living organisms). The subject may be a mammal, such as a human, a mouse, a rat, a hamster, a guinea pig, a rabbit, a dog, a cat, cattle, a goat, a sheep, a monkey, or a horse. Preferably, the subject suitable for use in the present method is a human. Depending on the type of biological sample, appropriate tools and/or procedures can be employed to obtain the isolated sample from the subject. Examples of isolated (biological) samples include, but are not limited to, whole blood samples, serum samples, plasma samples, urine samples, saliva samples, cerebrospinal fluid samples, synovial fluid samples, pleural fluid samples, peritoneal fluid samples, and lymphatic fluid samples. According to one working example, the isolated sample of the present disclosure is a urine sample.

According to embodiments of the present disclosure, the level of GM2A protein in the biological sample is determined by methods well known in the art, which include but are not limited to, enzyme-linked immunosorbent assay (ELISA), lateral flow immunoassay, western blotting, immunohistochemistry (IHC), mass spectrometry (MS), and etc. In some embodiments, the level of GM2A protein is determined by ELISA. Practically, by comparing the levels of GM2A protein that respectively detected in the control and isolated biological samples, or by determining the presence or absence of GM2A protein in the isolated biological sample (in which case, the control sample does not have GM2A), the present method is capable of determining whether the subject has a lung cancer or not. The control sample is derived from a healthy subject, or a subject having no lung cancers. When the level of GM2A protein in the isolated biological sample is higher than that in the control group sample, then the subject has lung cancer or is at risk of developing lung cancer.

According to preferred embodiments, the lung cancer is at an early stage.

By the virtue of the above features, the present recombinant antibody, kit, and method can provide early identification and detection of lung cancer, especially in a non-invasive manner, therefore enabling the identified patients to be treated properly.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### Example

Materials and Methods

Single B Cell Sorting and Cloning

Purified human GM2A protein with the amino acid sequence designated as SEQ ID NO: 5, was used as the antigen. GM2A was dissolved in phosphate-buffered saline (PBS) emulsified with Freund's adjuvant and was used for the first immunization of New Zealand white rabbits (approximately 6 to 9 weeks old), followed by a boosting immunization using Freund's incomplete adjuvant. Antisera were collected from the rabbit's ear veins, and the antibody titer was determined via ELISA. Rabbit splenocytes were harvested when the antisera exhibited the highest affinity. Subsequently, these splenocytes were sorted with the aid of biotinylated IgG1 using flow cytometry. The thousands of primary B cell strains thus obtained were individually seeded into 96-well plates (one cell per well) and cultured for two weeks in B cell culture media containing monoclonal rabbit IgG antibodies.

In second sorting, an ELISA assay kit coated with hIgG4 and purified cynolgG was used to identify and quantify B cell clones. The antigen-specific B cell clones were sorted and selected based on the ratio of positive to negative ELISA signals. Next, the gene sequences of these IgG heavy chains (VH) and light chains (VL) were amplified by RT-PCR. The PCR products of the heavy and light chains were transfected into HEK293F cells. Through enzyme immunoassays and molecular interactions, the specificity of binding of the transiently expressed recombinant rabbit IgG clones to hIgG1, hIgG4, and hFab was analyzed. Based on the ELISA results showing specific binding of hIgG1-hIgG4 and hFab, the PCR products from selected positive B cells (approximately 100 clones) were cloned into a mammalian expression vector, such as pcDNA3.1, to scale up antibody production in HEK293F cells. After purified by chromatography, the recombinant rabbit monoclonal antibody (rmAb) clones produced through HEK293F transfection was further evaluated. The clones with the highest affinity were selected, resulting in the production of the rabbit monoclonal recombinant antibody products of the present disclosure (i.e., anti-GM2A-I and anti-GM2A-II).

Detecting the Present Recombinant Protein by Sandwich ELISA

Horseradish peroxidase (HRP) was conjugated to the detection antibody using commercially available or conventional HRP-protein conjugation kits. Specifically, 50 μg of purified detection antibody (e.g., anti-GM2A-II antibody of the present disclosure) was added to the HRP mixture at a molar ratio of antibody to HRP of 1:2. The conjugating reaction was quenched according to the manufacturer's instructions. Thus, the HRP-detection antibody conjugate was produced.

A sandwich ELISA was performed using a 96-well microplate. At first, 100 μL of capture antibodies (i.e., the anti-GM2A-I antibody of the present disclosure) were coated onto each well of the 96-well microplate and incubated at 4° C. overnight. Subsequently, 100 μL of standard samples (containing known concentrations of GM2A protein) or samples obtained from subjects (with or without GM2A protein) were added to each well. After incubation in the dark at 37° C. for one hour, the microplate was washed twice with 200 μL of wash buffer. Following the washes, 100 μL of HRP-detection antibody conjugate (at a concentration of 0.025 μg/mL) was added to each well. The microplate was incubated in the dark at 37° C. for an additional hour. After another two washes with 200 μL of wash buffer, 100 μL of the substrate, 3,3′,5,5′-tetramethylbenzidine (TMB), was added to each well, allowing the reaction to proceed at room temperature in the dark for 5 minutes. 50 μL of 1 N H₂SO₄ was added to each well to terminate the chromogenic reaction and to develop color. The color intensity was measured by recording the absorbance of each well at a wavelength of 450 nm. These obtained absorbance values were then used to generate a calibration curve, establishing a correlation with known GM2A protein concentrations in the standard samples.

### Example 1: The Recombinant Antibodies, Anti-GM2A-I and Anti-GM2A-II of the Present Disclosure

Two highly specific and sensitive rabbit monoclonal antibodies against GM2A protein were produced by single B-cell sorting and cloning technology as described in the “Materials and Methods” section, and the thus obtained antibodies were respectively termed as “anti-GM2A-I” and “anti-GM2A-II”.

After gene sequencing, the amino acid sequences of the variable regions of anti-GM2A-1 and anti-GM2A-II are listed in Table 3, respectively, in which complementary determining regions (CDRs) are indicated in bold letters.

TABLE 3
Amino acid sequences of anti-GM2A-I and anti-GM2A-II of the present
disclosure

| | | | Sequence |
|---|---|---|---|
| | Variable | Amino acid sequence | identifiers |
| Antibody | region | (N-terminal → C-terminal) | (SEQ ID NO) |
| anti-GM2A-I | VL | LEYPFTFGAGTKLELKDIVMTQTASSVYVTP | 12 |
| | | GESVSISCRSTKSLLHSNGDTYLYWFLQRP | |
| | | GQSPQLLIYRMSNLAPGVPDRFSGGGSGTA | |
| | | FTLRISRVEAEDVGVYYCMQHI | |
| | VH | CEIQLQQSGALELVRPGTSVAKISCMASGYI | 13 |
| | | SFTGYNMNWRVRQSHGRSLEWIGNINPYF | |
| | | GSPNYNQKFKGKATLTLDRSSNTAYMQLN | |
| | | RVTSEDSAVYYCASRQLGLGDTMDYWGQ | |
| | | GTSVTVSS | |
| anti-GM2A-II | VL | LEYPFTFGGGTKLEIKDIVMTQSHKFMSTSV | 14 |
| | | GDRVTITCKASQDVSTAVGWYQQKPGQSP | |
| | | KLLIYWASTRHTGVPDRFTGSRFGTDYTLTI | |
| | | SSVQAEDLALYYCQQHYI | |
| | VH | CQIQLQQSGPLELGKPGASVAKISCKASDYI | |
| | | TFTDFYISWRVKQRPGQGLEWIGWIYPLDD | 15 |
| | | HTKYNEKFKGKATLTVDISSNTVYMQLSSL | |
| | | TSEDSAVYFCARIYGYAMDYWGQGTSVTV | |
| | | SS | |

### Example 2: Efficiency of the Present Anti-GM2A-I or Anti-GM2A-II Antibodies in the Detection of GM2A Protein

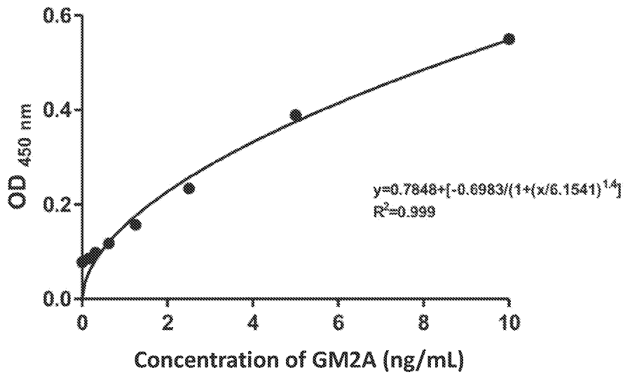

This example was to evaluate the binding affinity and specificity of the present recombinant antibody (i.e., anti-GM2A-I or anti-GM2A-II), against GM2A protein. To this purpose, the present anti-GM2A-I and anti-GM2A-II antibodies were respectively used as capture and detection antibodies in accordance with the procedures described in the “Materials and Methods” section above. As specific antibodies against GM2A protein were not commercially available at the time this study was conducted the control antibodies used in this study were antibody products from other positive B cells set forth in the “Materials and Methods” and “Example 1” sections. The results are summarized in FIG. 1, Table 4, and Table 5, respectively.

2.1 The Calibration Curve for GM2A Protein

The absorbance of GM2A protein standard captured by the present antibodies (anti-GM2A-I and anti-GM2A-II) at various concentrations at 450 nm were measured and results are summarized in Table 4, which were then used to construct a calibration curve for subsequent determination of the level of GM2A in a sample.

TABLE 4
Absorbance of GM2A protein standard captured
by the present anti-GM2A-I and anti-GM2A-II

| Reagent | Concentration | | Absorbance (O.D 450 nm) |
|---|---|---|---|
| Capture antibody | 1 | μg/mL | |
| (anti-GM2A-I) | | | |
| Detection antibody | 0.025 | μg/mL | |
| (anti-GM2A-II) | | | |
| GM2A protein standard | 0 | | 0.078 |
| | 0.156 | (ng/mL) | 0.086 |
| | 0.313 | (ng/mL) | 0.098 |
| | 0.625 | (ng/mL) | 0.118 |
| | 1.25 | (ng/mL) | 0.157 |
| | 2.5 | (ng/mL) | 0.234 |
| | 5 | (ng/mL) | 0.389 |
| | 10 | (ng/mL) | 0.549 |

The data in Table 4 indicates that the antibodies of the present disclosure were capable of detecting GM2A protein in trace amounts (0.025 or 1 μg/mL). Particularly, at low concentrations of GM2A protein, the present antibodies could distinguish samples that completely lacked expression of GM2A proteins from those expressing merely a trace amount of GM2A protein (e.g., 0.156 ng/mL). Compared to the control group (i.e., antibodies from other positive B cell clones from the same batch), the present antibodies could effectively identify trace amounts of GM2A protein at only one-fifth to one-half of the concentration of the control antibody (data not shown).

2.2 The Efficacy of the Present Antibodies in Identifying GM2A in Human Urine Samples

The efficacy of the present antibodies or kits in detecting GM2A protein in biological samples was investigated. To this purpose, urine samples were randomly collected from three healthy adult male and three healthy adult female volunteers (i.e., individuals with no lung cancer and no potential risk factors for developing lung cancer). ELISA for each samples were conducted using the present anti-GM2A-I and anti-GM2A-II antibodies. The concentration of GM2A protein in each urine samples was determined with the aid of the calibration curve constructed in Example 2.1. Results are summarized in Table 5.

TABLE 5
GM2A protein levels in human urine samples

| Absorbance | Individual number | | | | | |
|---|---|---|---|---|---|---|
| (O.D 450 nm) | 1 | 2 | 3 | 4 | 5 | 6 |
| Sample 1 | 0.147 | 0.112 | 0.082 | 0.076 | 0.126 | 0.111 |
| Sample 2 | 0.137 | 0.108 | 0.081 | 0.078 | 0.121 | 0.106 |
| Average* | 0.142 | 0.110 | 0.082 | 0.077 | 0.124 | 0.109 |
| GM2A concentration | 1.071 | 0.561 | 0.167 | 0.166 | 0.786 | 0.534 |
| (ng/mL)* | | | | | | |

*The average values are rounded to three decimal places.

The data in Table 5 indicates that the present antibodies can successfully detect trace amounts of GM2A protein in urine samples of healthy humans, even when the protein was present at concentrations lower than 1 ng/mL.

The data collectively confirmed that the recombinant antibodies provided herein can specifically and rapidly bind to GM2A, even in trace amounts of this protein. Further, owing to the non-invasive and high sensitivity characteristics of the present antibodies, it is possible to effectively distinguish healthy individuals from those having lung cancers, including subjects having an early stage lung cancer, so that appropriate treatment may be administered in a more timely manner.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A recombinant antibody or a fragment thereof comprising a light chain variable (VL) region and a heavy chain variable (VH) region, wherein the VL region comprises a first complementarity determining region (CDR-L1), a second complementarity determining region (CDR-L2), and a third complementarity determining region (CDR-L3), and the VH region comprises a first complementarity determining region (CDR-H1), a second complementarity determining region (CDR-H2), and a third complementarity determining region (CDR-H3), wherein the CDR-L1, the CDR-L2, the CDR-H1, the CDR-H2, and the CDR-H3 respectively comprise the amino acid sequences of SEQ ID NOs: 1, 2, 3, 4, and 5, and the CDR-L3 comprises the amino acid sequence of MQH; or
the CDR-L1, the CDR-L2, the CDR-L3, the CDR-H1, the CDR-H2, and the CDR-H3 respectively comprise the amino acid sequences of SEQ ID NOs: 6, 7, 8, 9, 10 and 11.

2. The recombinant antibody of claim 1, wherein
the VL region comprises an amino acid sequence at least 85% identical to SEQ ID NO: 12, and the VH region comprises an amino acid sequence at least 85% identical to SEQ ID NO: 13; or
the VL region comprises an amino acid sequence at least 85% identical to SEQ ID NO: 14, and the VH region comprises an amino acid sequence at least 85% identical to SEQ ID NO: 15.

3. The recombinant antibody of claim 1, wherein
the VL region comprises an amino acid sequence of SEQ ID NO: 12, and the VH region comprises amino acid sequence of SEQ ID NO: 13; or
the VL region comprises an amino acid sequence of SEQ ID NO: 14, and the VH region comprises an amino acid sequence of SEQ ID NO: 15.

4. A kit for detecting ganglioside GM2 activator (GM2A) in a biological sample, comprising
a first recombinant antibody and a second recombinant antibody, independently comprising a VL region and a VH region, wherein the VL region comprises a first complementarity determining region (CDR-L1), a second complementarity determining region (CDR-L2), and a third complementarity determining region (CDR-L3), and the VH region comprises a first complementarity determining region (CDR-H1), a second complementarity determining region (CDR-H2), and a third complementarity determining region (CDR-H3), wherein
the CDR-L1, the CDR-L2, the CDR-H1, the CDR-H2, and the CDR-H3 respectively comprise the amino acid sequences of SEQ ID NOs: 1, 2, 3, 4, and 5, and the CDR-L3 comprises the amino acid sequence of MQH; or
the CDR-L1, the CDR-L2, the CDR-L3, the CDR-H1, the CDR-H2, and the CDR-H3 respectively comprise the amino acid sequences of SEQ ID NOs: 6, 7, 8, 9, 10 and 11; and
a container containing the first and second recombinant antibodies.

5. The kit of claim 4, wherein
the VL region comprises an amino acid sequence at least 85% identical to SEQ ID NO: 12, and the VH region comprises an amino acid sequence at least 85% identical to SEQ ID NO: 13; and
the VL region comprises an amino acid sequence at least 85% identical to SEQ ID NO: 14, and the VH region comprises an amino acid sequence at least 85% identical to SEQ ID NO: 15.

6. The kit of claim 4, wherein
the VL region comprises an amino acid sequence of SEQ ID NO: 12, and the VH region comprises an amino acid sequence of SEQ ID NO: 13; and
the VL region comprises an amino acid sequence of SEQ ID NO: 14, and the VH region comprises an amino acid sequence of SEQ ID NO: 15.

7. A method for determining whether a subject has a lung cancer by use of a biological sample isolated from the subject comprising:
(a) detecting the level of ganglioside GM2 activator (GM2A) in the biological sample by use of the kit of claim 4; and
(b) comparing the level of GM2A in the biological sample of step (a) with that in a control sample, wherein the subject has the lung cancer when the detected level of GM2A in the biological sample is higher than that in the control sample.

8. The method of claim 7, wherein the biological sample is a urine sample.

9. The method of claim 8, wherein the subject is a human.

10. The method of claim 9, wherein the lung cancer is at an early stage.
